(19) United States
(12) Patent Application Publication
(10) Pub. No.: US 2021/0038922 A1
(43) Pub. Date: Feb. 11, 2021

(54) Inhomogeneous MRI System

(71) Applicant: The Board of Regents of the University of Texas System, Austin, TX (US)

(72) Inventors: Xun Jia, Garland, TX (US); Chenyang Shen, Dallas, TX (US); Nima Hassan-Rezaeian, Dallas, TX (US); Jace Gradinetti, Dallas, TX (US); Steve B. Jiang, Southlake, TX (US); Robert Timmerman, Austin, TX (US)

(73) Assignee: The Board of Regents of the University of Texas System, Austin, TX (US)

(21) Appl. No.: 16/943,507

(22) Filed: Jul. 30, 2020

Related U.S. Application Data
(60) Provisional application No. 62/882,692, filed on Aug. 5, 2019.

(51) Int. Cl. A61N 5/10; G01R 33/48; G01R 33/385; G01R 33/30; A61B 5/055; A61B 6/03; A61B 5/00; A61B 6/00

(52) U.S. Cl. CPC A61N 5/1081; G01R 33/4812; G01R 33/385; G01R 33/307; A61B 6/4085; A61N 5/107; A61B 6/032; A61B 5/0035; A61B 6/5247; A61B 5/055

(57) ABSTRACT

A system for MRI-guided radiotherapy is disclosed herein. The system includes a radiotherapy apparatus in the form of a linear accelerator or heavy ion system, an MRI portion, and a patient platform. The linear accelerator portion includes a stand, a gantry coupled to the stand, and a treatment head. The gantry is configured to rotate about the stand. The treatment head is coupled to the gantry. The treatment head is configured to deliver a radiotherapy beam. A system for MRI-guided radiotherapy is disclosed herein. The system includes a radiotherapy portion and an MRI portion adjacent to the radiotherapy portion. The MRI portion includes a magnet configured to generate an inhomogeneous magnetic field.

### CROSS-REFERENCE TO RELATED APPLICATIONS

This application claims priority to U.S. Application Ser. No. 62/882,692, filed Aug. 5, 2019, which is hereby incorporated by reference in its entirety.

### FIELD OF THE DISCLOSURE

The present disclosure generally relates to an inhomogeneous MRI system and integrations of an inhomogeneous MRI system with a radiotherapy apparatus

### BACKGROUND

Image guidance plays a critical role in radiotherapy to improve (e.g., ensure) treatment accuracy. Conventional approaches to such processes involve cone-beam computer tomography (CBCT) installed on a radiotherapy apparatus, e.g. a medical linear accelerator (LINAC). While CBCT can provide an x-ray attenuation image to guide patient positioning, low soft-tissue contrast affects the delineation of anatomical features, hindering setup accuracy in many cases.

Further, CBCT typically suffers from excessive imaging x-ray dose, low-soft tissue contrast, and lack of real-time imaging capability. Most recently, there has been success integrating conventional diagnostic MRI systems with a LINAC for MRI-based image guidance. However, the high development cost, mainly due to solving technical challenges of integrating MRI with LINAC under electromagnetic and geometric constraints, have led to high system costs, impeding clinical adoption of the MRI-LINAC systems.

### SUMMARY

In some embodiments, a system for MRI-guided radiotherapy is disclosed herein. The system includes a linear accelerator portion, an MRI portion, and a patient platform. The linear accelerator portion includes a stand, a gantry coupled to the stand, and a treatment head. The gantry is configured to rotate about the stand. The treatment head is coupled to the gantry. The treatment head is configured to deliver a radiotherapy beam. The MRI portion is adjacent to the linear accelerator portion. The MRI portion includes a magnet and one or more gradient coils. The magnet is configured to generate an inhomogeneous magnetic field. The one or more gradient coils is configured to generate one or more magnetic fields to distort the inhomogeneous magnetic field. The patient platform is configured to move between the linear accelerator portion and the MRI portion. The patient platform includes a patient receiving surface for supporting a patient during MRI-guided radiotherapy.

In some embodiments, the linear accelerator portion includes a cone-beam computer tomography (CBCT) imaging system coupled with the gantry.

In some embodiments, the CBCT imaging system is positioned substantially perpendicular to the MRI portion.

In some embodiments, the MRI portion further includes a body formed from a material configured to block magnetic waves and radiofrequency radiation. The magnet is disposed in an interior volume of the body.

In another embodiment, a system for MRI guided radiotherapy is disclosed herein. The system includes a linear accelerator and a retrofit MRI apparatus. The linear accelerator system is configured to deliver a radiotherapy beam to a patient. The retrofit MRI apparatus is coupled with the linear accelerator system. The retrofit MRI apparatus includes a magnet configured to generate an inhomogeneous magnetic field to image the patient.

In some embodiments, the linear accelerator system includes a stand, a gantry, and a treatment head. The gantry is coupled to the stand. The gantry is configured to rotate about the stand. The treatment head coupled to the gantry. The treatment head is configured to deliver the radiotherapy beam to the patient.

In some embodiments, the linear accelerator system further includes a positioning ring coupled to the gantry.

In some embodiments, the magnet is coupled with the positioning ring.

In some embodiments, the linear accelerator system further includes a cone-beam computer tomography imaging system coupled with the gantry.

In some embodiments, the retrofit MRI apparatus includes one or more gradient coils configured to generate a magnetic field that distorts the inhomogeneous magnetic field generated by the magnet.

In some embodiments, a system for MRI-guided radiotherapy using heavy ions, e.g. protons or heavier ions, is disclosed herein. The system includes a radiotherapy portion and an MRI portion. The radiotherapy portion includes a supporting system, a gantry, and a nozzle. The gantry is coupled to the supporting system. The gantry is configured to rotate about the supporting system. The nozzle is coupled to the gantry. The nozzle is configured to deliver charged particle radiation such as protons or heavier ions. The MRI portion is adjacent to the radiotherapy portion. The MRI portion includes a magnet, one or more gradient coils, and a radio frequency (RF) system. The magnet is configured to generate an inhomogeneous magnetic field. The one or more gradient coils are configured to generate one or more magnetic fields to distort the inhomogeneous magnetic field. The RF system is configured to send and receive RF electromagnetic waves for imaging purpose.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrated only typical embodiments of this disclosure and are therefore not to be considered limiting of its scope, for the disclosure may admit to other equally effective embodiments.

FIG. 1A is a block diagram illustrating an integrated inhomogeneous MRI-LINAC system, according to example embodiments.

FIG. 1B is a partial cross-sectional diagram illustrating the integrated inhomogeneous MRI-LINAC system of FIG. 1A, according to example embodiments.

FIG. 1C is a partial cross-sectional diagram illustrating the integrated inhomogeneous MRI-LINAC system of FIG. 1A, according to example embodiments.

FIG. 2A is a block diagram illustrating an integrated inhomogeneous MRI-LINAC system, according to example embodiments.

FIG. 2B is a block diagram illustrating the integrated inhomogeneous MRI-LINAC system of FIG. 2A, according to example embodiments.

FIG. 3 is a block diagram illustrating an integrated inhomogeneous MRI-heavy ion radiotherapy system, according to example embodiments.

FIG. 4 is a flow diagram illustrating a method of operating system, according to example embodiments

FIG. 5A illustrates an example system configuration for implementing various embodiments of the present technology, according to example embodiments.

FIG. 5B illustrates an example system configuration for implementing various embodiments of the present technology, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

One or more techniques disclosed herein integrates a non-conventional inhomogeneous MRI system with a LINAC for image guidance in radiotherapy. Inhomogeneous MRI technology, low field strength, and small field of view (FOV) are typically not favored in diagnostic radiology due to the low image quality and small FOV associated therewith. Homogeneity/inhomogeneity may refer to the uniformity of a magnetic field generated by the MRI system. For purposes of image guidance, however, there is typically a lower requirement on image quality and size of FOV. Accordingly, the limitations of inhomogeneous, low-field, and small FOV MRI technology in diagnostic radiology are not present for image guidance. Further, the use of inhomogeneous MRI technology, low-field strength, and small FOV reduces the overall size of the integrated system. Accordingly, the compact system design and low development cost of inhomogeneous MRI provides a favorable alternative for MRI-LINAC system.

One or more techniques disclosed herein also integrates a non-conventional inhomogeneous MRI system with a radiotherapy apparatus for heavy ion radiotherapy. Similar to the benefits described above for integration of an MRI system with a LINAC, the addition of the inhomogeneous MRI system with the radiotherapy apparatus reduces the overall size and cost of the integrated system.

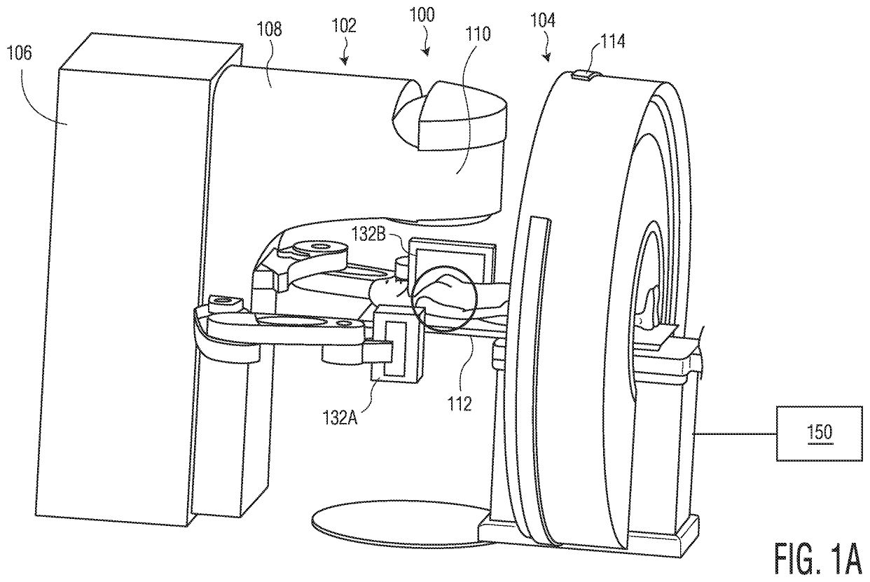

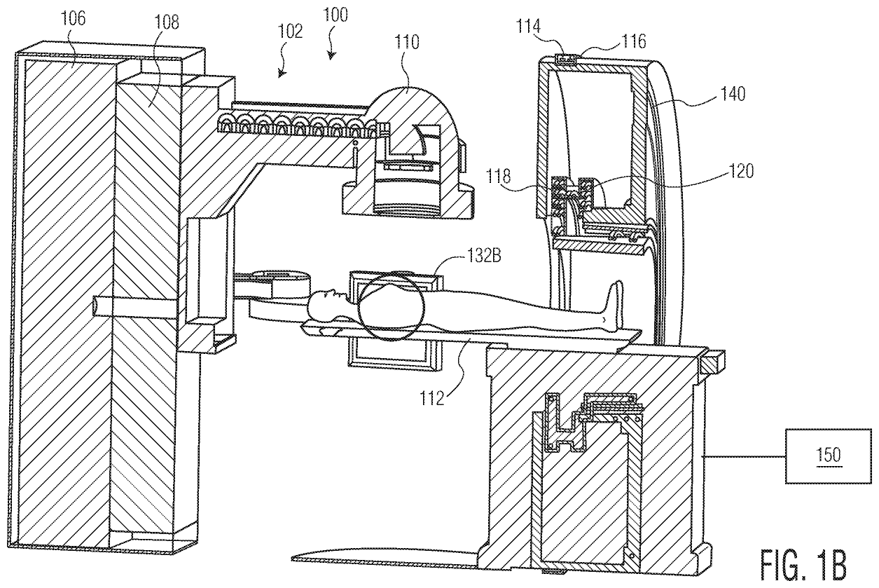

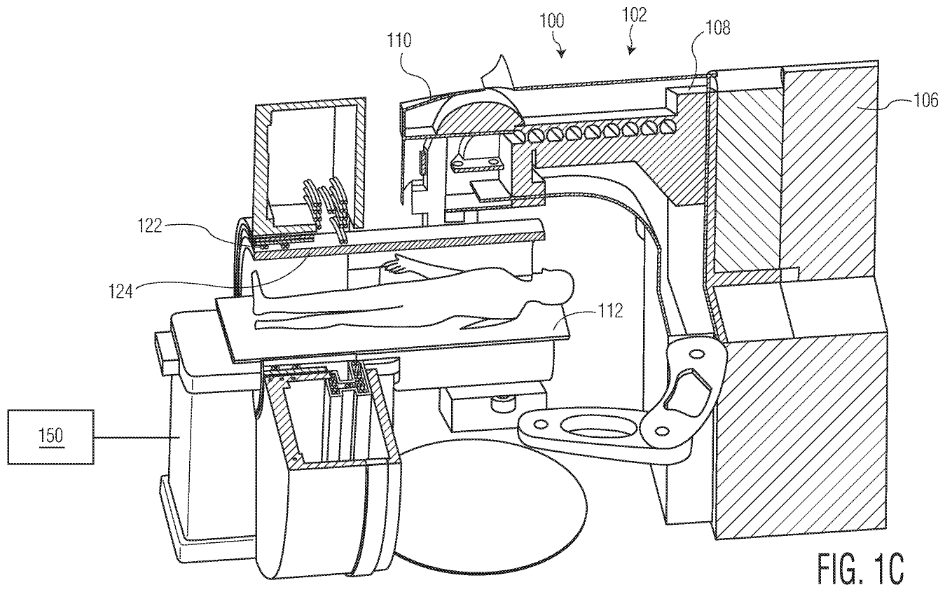

FIG. 1A is a block diagram illustrating an integrated inhomogeneous MRI-LINAC system 100 (hereinafter “system 100”), according to example embodiments. FIG. 1B is a partial cross-sectional diagram illustrating the integrated inhomogeneous MRI-LINAC system of FIG. 1A, according to example embodiments. As illustrated, system 100 includes a LINAC portion 102, an inhomogeneous MRI portion 104, and controller 150. LINAC portion 102 may include a stand 106, a gantry 108, a treatment head 110, and patient platform 112. Stand 106 may be coupled to gantry 108. In some embodiment, stand 106 may include one or more components for operating LINAC portion 102. Gantry 108 may be configured to rotate about an axis for delivering treatment to an individual from multiple angles. In other words, gantry 108 may be configured to rotate to aid in directing photons or electron beams to a patient. In some embodiments, gantry 108 may include treatment head 110. Treatment head 110 may be configured to shape and monitor photon or electron beams directed to the patient. Patient platform 112 may be configured to support a patient during treatment. In some embodiments, patient platform 112 may rotate about an axis. In some embodiments, patient platform 112 may move along a given axis.

In some embodiments, LINAC portion 102 may further include a CBCT x-ray imaging system 132A, 132B. In some embodiments, CBCT x-ray imaging system 132A, 132B and MRI portion 104 may be positioned substantially perpendicular to each other and configured such that any interference from each system is reduced (e.g., minimized).

Inhomogeneous MRI portion 104 may be positioned adjacent LINAC portion 102. MRI portion 104 may include a body 140 defining an interior volume (not shown). As illustrated, body 140 may be ring-shaped, i.e., body 140 may include an opening formed therein. Opening may allow for passage of patient platform 112 during operation. In some embodiments, MRI portion 104 may further include a magnet 114. Magnet 114 may be representative of a single magnet or multiple magnets. Generally, because an inhomogeneous magnetic field is to be generated by MRI portion 104 during operation, magnet 114 may be representative of a simple magnet design, opposed to more complex magnet designs utilized to achieve field homogeneity. Without requirement on field homogeneity, the magnet can be designed with a large freedom to meet compatibility requirement with LINAC. Magnet design can be achieved via standard optimization techniques to yield a sufficiently high, e.g. 0.3 T inhomogeneous field in the target region (circle) and a sufficiently low field at the magnetically sensitive regions of a LINAC, e.g. x-ray target and multi-leaf collimator. Magnet 114 may be made of permanent magnet, resistive magnet, or superconducting magnet. In the case of superconducting magnet, it may include one or more high temperature superconducting wires 116. In operation, magnet 114 may be configured to generate an inhomogeneous field for image guidance. For example, magnet 114 may generate the inhomogeneous magnetic field around the target treatment area of LINAC portion 102 for imaging purposes.

In some embodiments, inhomogeneous MRI portion 104 may further include superconducting wires 118. The superconducting wires may provide high current to generate a magnetic field. Locations, sizes, and currents of the wires may be designed to generate the magnetic field that is sufficient for MRI imaging at the target region and is low at the magnetically sensitive regions of a LINAC. Local magnet shielding around the magnetically sensitive regions of a LINAC may be performed to further reduce magnet field strength in these regions. Superconducting wires 118 may be surrounded by housing 120. Housing 120 may be configured to cool superconducting wires 118 inside housing 120. In some embodiments, housing 120 may be representative of a cryostat housing.

FIG. 1C is a partial cross-sectional diagram illustrating system 100, according to example embodiments. As shown, a cylinder is placed around the patient to help illustrate locations of gradient coils and RF coils (discussed below). In some embodiments, inhomogeneous MRI portion 104 may further include gradient coils 122. Gradient coils 122 may be configured to generate a magnetic field that slightly distorts the magnetic field generated by magnet 114. The shape, location, current, and other properties of the gradient coils may be designed following standard MRI gradient coil design approaches, e.g. target field method. One gradient coil can generate a spatially dependent magnetic field, based on which location information can be inferred in MR imaging process. Different techniques may be used to encode three directions (x-, y-, or z-directions) for three-dimensional imaging purpose. For example, gradient coils 122 may include at least one coil configured to encode one of three directions (e.g., one of the x-, y-, or z-direction). In such example, one of the other directions would be encoded by the inhomogeneous magnetic field generated by magnet 114 and the other direction would be encoded using a combination of the single coil and a mechanical rotation. In another example, gradient coils 122 may include a first gradient coil dedicated for encoding a first direction and a second gradient coil dedicated for encoding a second direction. In such example, the inhomogeneous magnetic field generated by magnet 114 would encode the third direction.

In some embodiments, inhomogeneous MRI portion 104 may further include radiofrequency (RF) coils 124. RF coils 124 may send and receive RF electromagnetic waves for MRI data acquisition. RF coils may be configured in standard MRI coil forms, e.g. body coil, surface coil, birdcage coil, or coil array.

In operation, MRI portion 104 may utilize an imaging sequence applicable for inhomogeneous fields. For example, MRI portion may utilize a spin-echo Carr-Purcell-Meiboom-Gill (CPMG) sequence.

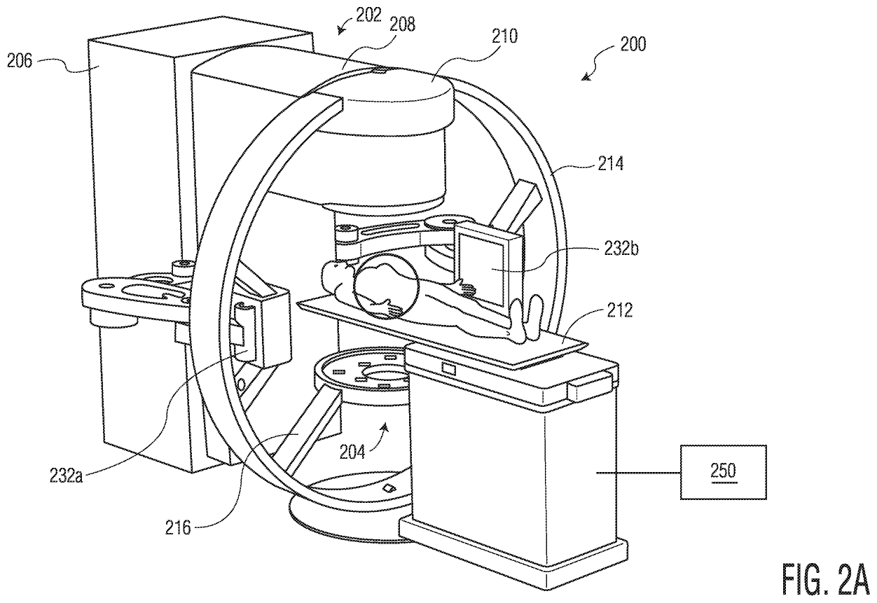

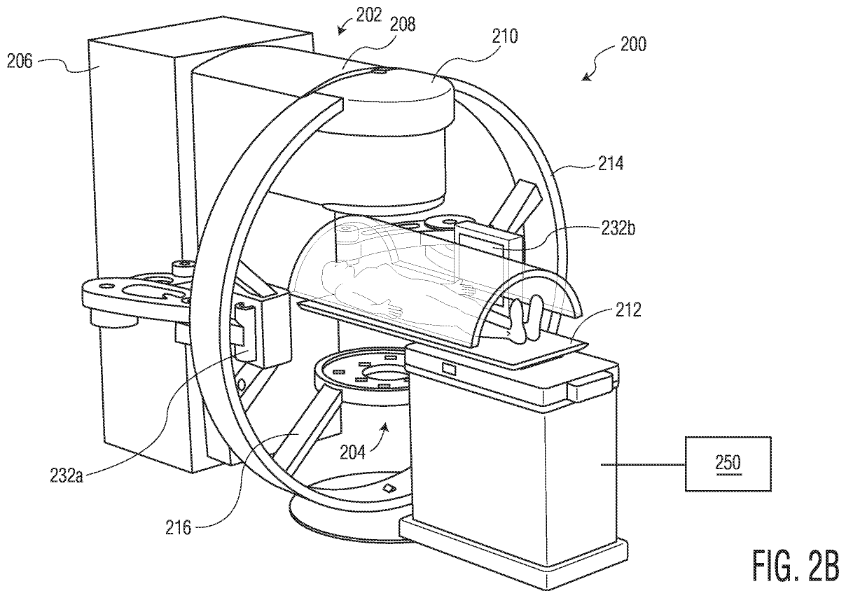

FIG. 2A is a block diagram illustrating an inhomogeneous MRI-LINAC system 200 (hereinafter “system 200”), according to example embodiments. FIG. 2B is a block diagram illustrating system 200, according to example embodiments. System 200 may include an existing LINAC portion 202, an inhomogeneous MRI portion 204, and controller 250. Inhomogeneous MRI portion 204 may be added to the existing LINAC portion 202 to form the retrofit system 200. For example, in some embodiments, MRI portion 204 may be added to existing LINAC machines that include, but are not limited to, TrueBeam® commercially available from Varian Medical Systems, Inc., VitalBeam® commercially available from Varian Medical Systems, Inc., Halcyon™ commercially available from Varian Medical Systems, Inc., Versa HD™ commercially available from Elekta, and Tomotherapy® commercially available from Accuray.

LINAC portion 202 may be representative of any LINAC system. LINAC portion 202 may include, for example, stand 206, gantry 208, treatment head 210, patient platform 212, and positioning ring 214. Stand 206 may be coupled to gantry 208. In some embodiment, stand 206 may include one or more components for operating LINAC portion 202. Gantry 208 may be configured to rotate about an axis for delivering treatment to an individual from multiple angles. In other words, gantry 208 may be configured to rotate to aid in directing photons or electron beams to a patient. In some embodiments, gantry 208 may include treatment head 210. Treatment head 210 may be configured to shape and monitor photon or electron beams directed to the patient. Patient platform 212 may be configured to support a patient during treatment. In some embodiments, patient platform 212 may rotate about an axis. In some embodiments, patient platform 212 may move along a given axis. Positioning ring 214 may be positioned about gantry 208. For example, as illustrated, positioning ring 214 may be positioned about gantry 208 and treatment head 210.

In some embodiments, LINAC portion 202 may further include a CBCT x-ray imaging system 232a, 232b. In some embodiments, CBCT x-ray imaging system 232a, 232b and MRI portion 204 may be positioned substantially perpendicular to each other and configured such that any interference from each system is reduced (e.g., minimized).

Similar to system 100 above, inhomogeneous MRI portion 204 may be selectively positioned adjacent LINAC portion 202. In some embodiments, inhomogeneous MRI portion 204 may be selectively mounted on positioning ring 214. MRI portion 204 may include a magnet 216. Magnet 216 may be representative of a single sided magnet or a few magnets. Because an inhomogeneous magnetic field is to be generated by MRI portion 204 during operation, magnet 216 may be representative of a simple magnet design, opposed to more complex magnet designs utilized to achieve field homogeneity. Without requirement on field homogeneity, the magnet can be designed with a large freedom to meet compatibility requirement with LINAC. Magnet design can be achieved via standard optimization techniques to yield a sufficiently high, e.g. 0.3 T inhomogeneous field in the target region (circle) and a sufficiently low field at the magnetically sensitive regions of a LINAC, e.g. x-ray target and multi-leaf collimator. In some embodiments, magnet 216 may be made of permanent magnet, resistive magnet, or superconducting magnet. In the case of superconducting magnet, it may include one or more high temperature superconducting wires (not shown). In operation, magnet 216 may be configured to generate an inhomogeneous field for image guidance. For example, magnet 216 may generate an inhomogeneous magnetic field around the target treatment area of an individual on patient platform 212 for imaging purposes.

Generally, as illustrated in FIG. 2A and FIG. 2B, magnet 216 may be positioned away from patient platform 212. Positioning magnet 216 away from patient platform 212 allows patient platform 212 to rotate.

In some embodiments, inhomogeneous MRI portion 204 may further include gradient coils (not shown). Gradient coils may be configured to generate a magnetic field that slightly distorts the inhomogeneous magnetic field generated by magnet 216. The shape, location, current, and other properties of the gradient coils may be designed following standard MRI gradient coil design approaches, e.g. target field method. One gradient coil can generate a spatially dependent magnetic field, based on which location information can be inferred in MR imaging process. Different techniques may be used to encode three directions (x-, y-, or z-directions) for three-dimensional imaging purpose. For example, gradient coils may include at least one coil configured to encode one of three directions (e.g., one of the x-, y-, or z-directions). In such example, one of the other directions would be encoded by the inhomogeneous magnetic field generated by magnet 216 and the other direction would be encoded using a combination of the single coil and a mechanical rotation. In another example, gradient coils may include a first gradient coil dedicated for encoding a first direction and a second gradient coil dedicated for encoding a second direction. In such example, the inhomogeneous magnetic field generated by magnet 216 would encode the third direction.

In some embodiments, inhomogeneous MRI portion 104 may further include radiofrequency (RF) coils. The RF coils send and receive RF electromagnetic waves for MRI data acquisition. The RF coils may be configured in standard MRI coil forms, e.g. body coil, surface coil, birdcage coil, or coil array. The RF coils can be designed following standard MRI coil design methods.

In some embodiments, during operation, MRI portion 204 may utilize an imaging sequence applicable for inhomogeneous fields. For example, MRI portion 204 may utilize a spin-echo Carr-Purcell-Meiboom-Gill (CPMG) sequence.

Before the treatment delivery, a patient may be position on the treatment platform. The MRI data acquisition is performed to acquire MR images in the targeted region. The image field of view may be small but sufficient to cover the tumor region that is clinically important. The image field of view may be smaller than the entire patient body. CBCT may be used to image the remaining regions to provide anatomical information complement to MRI for different purposes, e.g. generate a new treatment plan. Based on the acquired images, the patient platform may be moved to position the tumor accurately with the treatment beam. A treatment replanning may be performed to generate a new treatment plan based on the acquired patient anatomy. After that, the treatment is delivered. During treatment delivery, MRI may be performed to monitor patient body motion to ensure accuracy of treatment delivery and patient safety, e.g. interrupting delivery if the tumor is found moving away from the planned region largely.

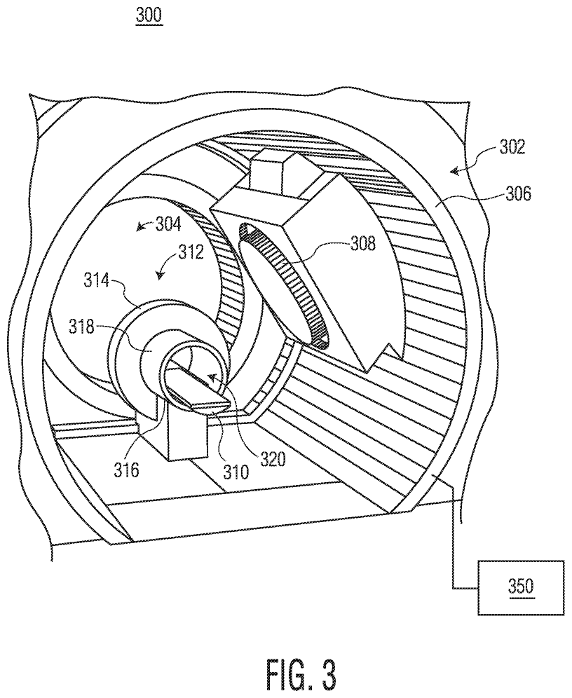

FIG. 3 is a block diagram illustrating an integrated inhomogeneous MRI-heavy ion radiotherapy system 300 (hereinafter “system 300”), according to example embodiments. As illustrated, system 300 may include radiotherapy portion 302, MRI portion 304, and controller 350. Radiotherapy portion 302 may be representative of an apparatus configured to deliver heavy charged particles that are accelerated by an accelerator. In some embodiments, the heavy charged particles may be representative of heavy ions, such as, but not limited to, protons and heavy ions (e.g., helium, carbon, etc.).

Radiotherapy portion 302 may include a support system (not shown), a gantry 306, a treatment nozzle 308, and a patient platform 310. Gantry 306 may be coupled to the support. In some embodiment, the support may include one or more components for operating radiotherapy portion 302. Gantry 306 may be configured to rotate about an axis for delivering treatment to an individual from multiple angles. In some embodiments, gantry 306 may include nozzle 308. Nozzle 308 may be configured to deliver heavy ion particles to a patient positioned on patient platform 310. For example, in operation, while gantry 306 rotates about its axis, nozzle 308 may be configured to deliver heavy ion particles to the patient. In some embodiments, radiotherapy portion 302 may accelerate the heavy charged particles through an accelerator (not shown) prior to delivery by nozzle 308. Patient platform 310 may be configured to support a patient during treatment. In some embodiments, patient platform 310 may rotate about an axis. In some embodiments, patient platform 310 may move along a given axis.

Generally, image guidance plays an important role in heavy ion radiotherapy to ensure treatment accuracy. Similar to use in the LINAC system described above, an inhomogeneous MRI scanner can be designed as part of the delivery system of the heavy ion therapy delivery system or be retrofit to existing heavy ion therapy delivery systems. The use of inhomogeneous MRI technology, low-field strength, and small FOV reduces the overall size of the integrated system and the electronic conflicts between the MRI system and the radiotherapy system.

Inhomogeneous MRI portion 304 may be positioned adjacent radiotherapy portion 302. MRI portion 304 may include a body 312 defining an interior volume (not shown). As illustrated, body 312 may be ring-shaped, i.e., body 312 may include an opening 320 formed therein. Opening 320 may allow for passage of patient platform 310 during operation. In some embodiments, body 312 may be formed from a material configured to block radiofrequency electromagnetic waves to ensure image quality. In some embodiments, MRI portion 304 may further include a magnet 314 and one or more gradient coils 316. Magnet 314 may be representative of a single magnet or multiple magnets.

Generally, because an inhomogeneous magnetic field is to be generated by MRI portion 304 during operation, magnet 314 may be representative of a simple magnet design, opposed to more complex magnet designs utilized to achieve field homogeneity. Without requirement on field homogeneity, the magnet can be designed with a large freedom to meet compatibility requirement with radiotherapy portion 302. Magnet 314 may be made of permanent magnet, resistive magnet, or superconducting magnet. In operation, magnet 314 may be configured to generate an inhomogeneous field for image guidance. For example, magnet 314 may generate the inhomogeneous magnetic field around the target treatment area of radiotherapy portion 302 for imaging purposes.

In some embodiments, magnet 314 may be located on the onside of the treatment beam to avoid blocking the therapeutic beam when delivered by nozzle 308. Magnet 314 may generate an inhomogeneous magnetic field inside a region of interest around the treatment iso center. One or more gradient coils 316 may be configured to generate one or more magnetic fields for spatial encoding purpose.

In some embodiments, MRI portion 304 may further include RF coils 318. RF coils 318 may be configured to send and receive RF electromagnetic waves for MRI data acquisition. RF coils 318 may be configured in standard MRI coil forms, e.g. body coil, surface coil, birdcage coil (shown in the figure), or coil array.

In some embodiments, the magnetic field generated by magnet 314 may perturb the trajectories of heavy ion beams. In some embodiments, the magnetic field distribution in the three-dimensional space may be known by measurement, once the MRI is installed on the heavy ion radiotherapy system. The perturbation may be computed based on physics principles and the known magnetic field distribution. The dose calculation engine in the treatment planning system of the heavy ion therapy should be modified to accommodate this perturbation. Advanced dose calculation method in the dose engine such as Monte Carlo simulations may be used to compute the dose distribution by the heavy ion beam under the effect of the magnetic field. The treatment plan may be modified based on the calculated dose distribution to achieve the treatment objectives under the effect of the magnetic field.

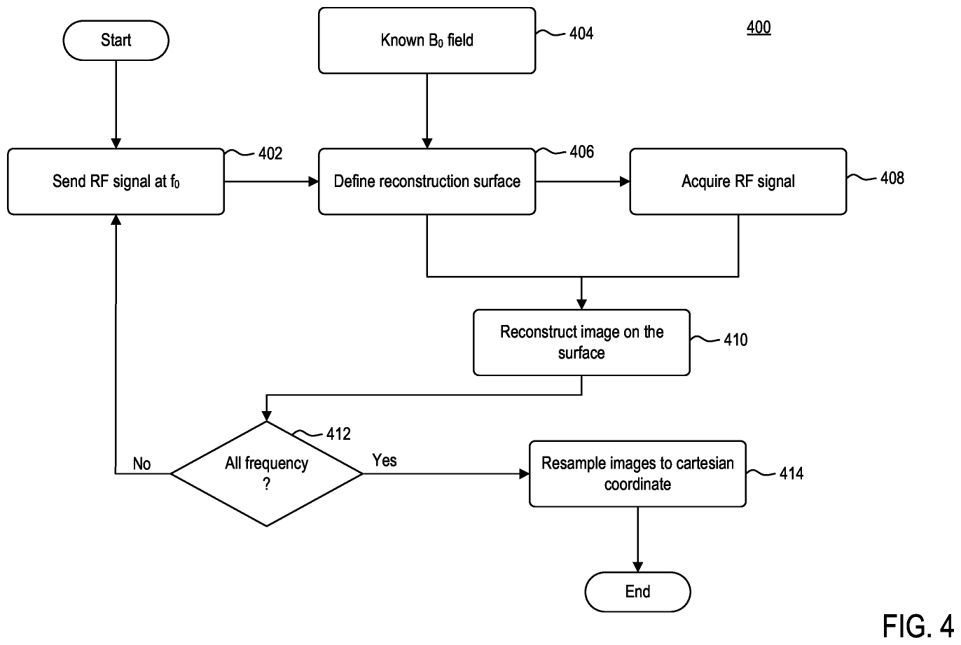

FIG. 4 is a flow diagram illustrating a method 400 of operating any of systems 100-300, according to example embodiments. For purposes of discussion, method 400 is discussed in conjunction with system 100. Method 400 may begin at step 402.

At step 402, system 100 may transmit an RF signal. For example, controller 150 may transmit an RF signal having a frequency, f₀. In some embodiments, the signal may be sent through an RF coil 124. RF signal may induce magnetic nuclear resonance in the imaging region.

At step 404, system 100 may input the known magnetic field distribution B₀ to the system. In some embodiments, the 3D distribution of the field B₀ may depend on the specific magnet design and construction. For a given magnet, the distribution can be known, for example by performing a measurement.

At step 406, system 100 may define a reconstruction surface given the RF f₀ signal and magnetic field B₀. For example, for a given frequency f₀ and magnetic field B₀, controller 150 may define a two-dimensional surface in space. In some embodiments, the two-dimensional surface in space may be defined based on the relationship γf₀=B(x, y, z), i.e., the iso-magnetic field surface corresponding to the frequency f₀.

At step 408, system 100 may acquire an RF signal. In some embodiments, the RF signal in the imaging region after the resonance may be received by the RF coil 124.

At step 410, system 100 may reconstruct an image on a surface of an object given the defined reconstruction surface and the acquired RF signal. For example, once the RF signal is sent to the field of view, it may excite spins on this two-dimensional surface. The signal received by RF coil 124 may contain excitation information in this surface, which can be used to reconstruct the image of this surface.

To obtain the volume image inside the imaging region, images on a number of surfaces may be needed. Each scan with frequency f₀ may provide an image on one surface. The scan may be repeated with different frequencies to acquire images for different surfaces. At step 412, system 100 may determine whether all frequencies have been considered. If, at step, 412, system determines that not all frequencies were considered, then method 400 reverts to step 402 and a new f₀ is selected. If, however, at step 412, system 100 determines that all frequencies were considered, then method 400 proceeds to step 414.

At step 414, system 100 may resample the images to cartesian coordinates. For example, using two-dimensional spatial encoding techniques achieved via gradient coils 122, a typical form of received RF signal may be expressed as

S=∫dxdyM(x, y, z(x, y))e^{i(k}^{ₓ}^{x+k}^{_{y}}^{y)}

where z(x, y) parametrizes the surface defined implicitly by γf₀=B(x, y, z). The acquired RF signals may be used to reconstruct an image defined on a two-dimensional surface using standard Fourier Transform algorithm, iterative algorithms, or other advanced algorithms.

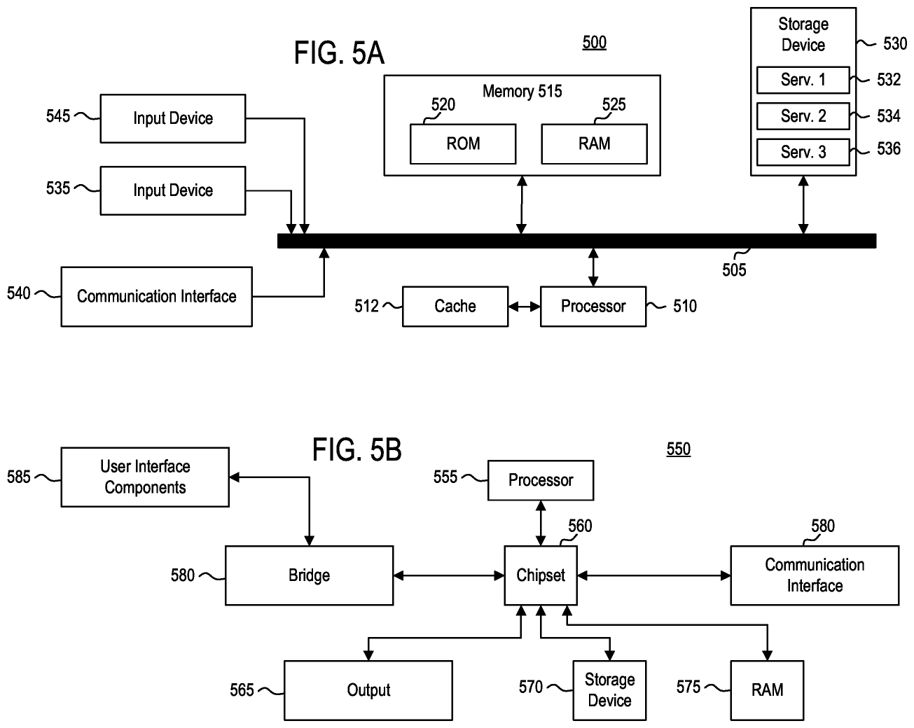

FIG. 5A illustrates a system bus computing system architecture 500, according to example embodiments. One or more components of system 500 may be in electrical communication with each other using a bus 505. System 500 may include a processor (e.g., one or more CPUs, GPUs or other types of processors) 510 and a system bus 505 that couples various system components including the system memory 515, such as read only memory (ROM) 520 and random access memory (RAM) 525, to processor 510. System 500 can include a cache of high-speed memory connected directly with, in close proximity to, or integrated as part of processor 510. System 500 can copy data from memory 515 and/or storage device 530 to cache 512 for quick access by processor 510. In this way, cache 512 may provide a performance boost that avoids processor 510 delays while waiting for data. These and other modules can control or be configured to control processor 510 to perform various actions. Other system memory 515 may be available for use as well. Memory 515 may include multiple different types of memory with different performance characteristics. Processor 510 may be representative of a single processor or multiple processors. Processor 510 can include one or more of a general purpose processor or a hardware module or software module, such as service 1 532, service 2 534, and service 3 536 stored in storage device 530, configured to control processor 510, as well as a special-purpose processor where software instructions are incorporated into the actual processor design. Processor 510 may essentially be a completely self-contained computing system, containing multiple cores or processors, a bus, memory controller, cache, etc. A multi-core processor may be symmetric or asymmetric.

To enable user interaction with the computing device 500, an input device 545 which can be any number of input mechanisms, such as a microphone for speech, a touch-sensitive screen for gesture or graphical input, keyboard, mouse, motion input, speech and so forth. An output device 535 can also be one or more of a number of output mechanisms known to those of skill in the art. In some instances, multimodal systems can enable a user to provide multiple types of input to communicate with computing device 500. Communications interface 540 can generally govern and manage the user input and system output. There is no restriction on operating on any particular hardware arrangement and therefore the basic features here may easily be substituted for improved hardware or firmware arrangements as they are developed.

Storage device 530 may be a non-volatile memory and can be a hard disk or other types of computer readable media that can store data that are accessible by a computer, such as magnetic cassettes, flash memory cards, solid state memory devices, digital versatile disks, cartridges, random access memories (RAMs) 525, read only memory (ROM) 520, and hybrids thereof.

Storage device 530 can include services 532, 534, and 536 for controlling the processor 510. Other hardware or software modules are contemplated. Storage device 530 can be connected to system bus 505. In one aspect, a hardware module that performs a particular function can include the software component stored in a computer-readable medium in connection with the necessary hardware components, such as processor 510, bus 505, display 535, and so forth, to carry out the function.

FIG. 5B illustrates a computer system 550 having a chipset architecture that can be used in operating system 100. Computer system 550 may be an example of computer hardware, software, and firmware that can be used to implement the disclosed technology. System 550 can include one or more processors 555, representative of any number of physically and/or logically distinct resources capable of executing software, firmware, and hardware configured to perform identified computations. One or more processors 555 can communicate with a chipset 560 that can control input to and output from one or more processors 555. In this example, chipset 560 outputs information to output 565, such as a display, and can read and write information to storage device 570, which can include magnetic media, and solid state media, for example. Chipset 560 can also read data from and write data to RAM 575. A bridge 580 for interfacing with a variety of user interface components 585 can be provided for interfacing with chipset 560. Such user interface components 585 can include a keyboard, a microphone, touch detection and processing circuitry, a pointing device, such as a mouse, and so on. In general, inputs to system 550 can come from any of a variety of sources, machine generated and/or human generated.

Chipset 560 can also interface with one or more communication interfaces 590 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by one or more processors 555 analyzing data stored in storage 570 or 575. Further, the machine can receive inputs from a user through user interface components 585 and execute appropriate functions, such as browsing functions by interpreting these inputs using one or more processors 555.

It can be appreciated that example systems 500 and 550 can have more than one processor 510 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A system for MRI-guided radiotherapy, comprising:
a linear accelerator portion comprising:
a stand;
a gantry coupled to the stand, the gantry configured to rotate about the stand; and
a treatment head coupled to the gantry, the treatment head configured to deliver a radiotherapy beam;
an MRI portion adjacent to the linear accelerator portion, the MRI portion comprising:
a magnet configured to generate an inhomogeneous magnetic field; and
one or more gradient coils configured to generate one or more magnetic fields to distort the inhomogeneous magnetic field; and
a radio frequency (RF) system to send and receive RF electromagnetic waves for imaging purpose; and
a patient platform configured to move between the linear accelerator portion and the MRI portion, the patient platform comprising a patient receiving surface for supporting a patient during MRI-guided radiotherapy.

2. The system of claim 1, wherein the linear accelerator portion comprises:
a cone-beam computer tomography (CBCT) imaging system coupled with the gantry.

3. The system of claim 2, wherein the CBCT imaging system is positioned substantially perpendicular to the MRI portion.

4. A system for MRI guided radiotherapy, comprising:
a linear accelerator system for delivering a radiotherapy beam to a patient; and
a retrofit MRI apparatus coupled with the linear accelerator system, the retrofit MRI apparatus comprising a magnet configured to generate an inhomogeneous magnetic field to image the patient.

5. The system of claim 4, wherein the linear accelerator system comprises:
a stand;
a gantry coupled to the stand, the gantry configured to rotate about the stand; and
a treatment head coupled to the gantry, the treatment head configured to deliver the radiotherapy beam to the patient.

6. The system of claim 5, wherein the linear accelerator system further comprising:
a positioning ring coupled to the gantry.

7. The system of claim 6, wherein the magnet is coupled with the positioning ring.

8. The system of claim 5, wherein the linear accelerator system further comprises:
a cone-beam computer tomography imaging system coupled with the gantry.

9. The system of claim 4, wherein the retrofit MRI apparatus comprises:
one or more gradient coils configured to generate a magnetic field that distorts the inhomogeneous magnetic field generated by the magnet; and
a radio frequency (RF) coil.

10. A system for MRI-guided radiotherapy, comprising:
a radiotherapy portion comprising:
a supporting system;
a gantry coupled to the supporting system, the gantry configured to rotate about the supporting system; and
a nozzle coupled to the gantry, the nozzle configured to deliver heavy charged particles; and
an MRI portion adjacent to the radiotherapy portion, the MRI portion comprising:
a magnet configured to generate an inhomogeneous magnetic field; and
one or more gradient coils configured to generate one or more magnetic fields to distort the inhomogeneous magnetic field; and
a radio frequency (RF) system to send and receive RF electromagnetic waves for imaging purpose.
